(19) United States
(12) Patent Application Publication
(10) Pub. No.: US 2021/0308425 A1
(43) Pub. Date: Oct. 7, 2021

(54) MEDICAL SHEATH ASSEMBLY

(71) Applicant: Baylis Medical Company Inc., Montreal (CA)

(72) Inventors: Jackie Leung, Richmond Hill (CA); Gareth Davies, Toronto (CA)

(21) Appl. No.: 17/222,549

(22) Filed: Apr. 5, 2021

Related U.S. Application Data
(60) Provisional application No. 63/006,363, filed on Apr. 7, 2020.

(51) Int. Cl. A61M 25/01

(52) U.S. Cl. CPC A61M 25/0158; A61M 2205/02; A61M 2025/0166

(57) ABSTRACT

A medical sheath assembly is configured to be positioned in a patient, and is also configured to convey, at least in part, an electromagnetic-transmission signal through the medical sheath assembly. The medical sheath assembly includes a sheath-support component being sympathetic, at least in part, for transmission of electromagnetic energy traveling through the sheath-support component, dispatched toward, or dispatched away from, the medical sheath assembly.

### TECHNICAL FIELD

This document relates to the technical field of (and is not limited to) (A) a medical sheath assembly including a sheath-support component, and/or method therefor, and/or (B) an electro-anatomical mapping system, a sensor assembly, and a medical sheath assembly including a sheath-support component, and/or method therefor.

### BACKGROUND

In medicine, a catheter, or sheath, is a thin tube made from medical grade materials serving a broad range of functions. Catheters are medical devices that can be inserted (at least in part) into the body to treat diseases or perform a surgical procedure.

### SUMMARY

It will be appreciated that there exists a need to mitigate (at least in part) at least one problem associated with the existing or known sheath assemblies (also called the existing technology). After much study of, and experimentation with, the existing or known sheath assemblies, an understanding (at least in part) of the problem and its solution have been identified (at least in part) and are articulated (at least in part) as follows:

Cardiac catheterization is a procedure to examine how well your heart is working. A thin, hollow tube called a catheter is inserted (at least in part) into a large blood vessel that leads to the heart of a patient. A cardiac catheter provides information on how well the heart works, identifies problems and allows for procedures to open blocked arteries, etc. A doctor may make a needle puncture through the skin and into a large blood vessel. A small straw-sized tube (such as, a sheath assembly or a catheter assembly) may be inserted (at least in part) into the vessel of a patient. The doctor may (gently) guide a catheter assembly (a long, thin tube) into the vessel through the sheath assembly. A video screen may show the position of the catheter assembly as the catheter assembly is threaded through the major blood vessels and to the heart of the patient.

For instance, electro-anatomical mapping (EAM) systems are configured to enable real-time (near real-time) visualization (three-dimensional or 3D) of intravascular catheters in the heart without exposure to radiation. In the case of EAM systems using electromagnetic fields, sensor coils (positioned to a catheter) are configured to receive electromagnetic signals from fixed sources positioned outside the body (of the patient) to triangulate the position of the medical assembly (such as a sheath assembly, etc.). However, for the case where a catheter is positioned inside a sheath assembly (in which the sheath assembly is braided with metal), the coil (sensor) becomes shielded from (and prevented from receiving) a signal transmitted form the electro-anatomical mapping (EAM) system. It may be appreciated that most metallic surfaces tend to interfere with the transmission of electromagnetic energy (such as radio frequency fields, etc.).

To mitigate, at least in part, at least one problem associated with the existing technology, there is provided (in accordance with a major aspect) an apparatus. The apparatus includes and is not limited to (comprises) a medical sheath assembly configured to be positioned in a patient. The medical sheath assembly is also configured to convey, at least in part, an electromagnetic-transmission signal through the medical sheath assembly. The medical sheath assembly includes a sheath-support component. Preferably, the sheath-support component does not interfere (in a significant way) with the transmission (conveyance) of electromagnetic energy traveling through the medical sheath assembly. Preferably, the sheath-support component is sympathetic (compatible, permissible), at least in part, for transmission (conveyance) of electromagnetic energy traveling (moving) through the sheath-support component, dispatched toward, or dispatched away from, the medical sheath assembly.

To mitigate, at least in part, at least one problem associated with the existing technology, there is provided (in accordance with a major aspect) an apparatus. The apparatus is for use with a sensor assembly configured to receive an electromagnetic-transmission signal emanating from an electro-anatomical mapping system. The sensor assembly is also configured to transmit a detected signal back to the electro-anatomical mapping system. The apparatus includes and is not limited to (comprises) a medical sheath assembly. The medical sheath assembly is configured to be positioned in a patient. The medical sheath assembly is also configured to receive the sensor assembly therein. The medical sheath assembly is also configured to convey, at least in part, the electromagnetic-transmission signal emanating from the electro-anatomical mapping system toward the sensor assembly positioned within the medical sheath assembly. The medical sheath assembly includes a sheath-support component. Preferably, the sheath-support component does not interfere (in a significant way) with the transmission (conveyance) of electromagnetic energy traveling through the medical sheath assembly. Preferably, the sheath-support component is sympathetic (compatible), at least in part, for transmission (conveyance) of electromagnetic energy traveling (moving) through the sheath-support component, dispatched toward, or dispatched away from, the medical sheath assembly.

To mitigate, at least in part, at least one problem associated with the existing technology, there is provided (in accordance with a major aspect) an apparatus. The apparatus includes and is not limited to (comprises) an electro-anatomical mapping system configured to transmit an electromagnetic-transmission signal. A sensor assembly is configured to receive the electromagnetic-transmission signal emanating from the electro-anatomical mapping system. The sensor assembly is also configured to transmit a detected signal back to the electro-anatomical mapping system. A medical sheath assembly is configured to be positioned in a patient. The medical sheath assembly is also configured to receive the sensor assembly therein. The medical sheath assembly is also configured to convey, at least in part, the electromagnetic-transmission signal emanating from the electro-anatomical mapping system toward the sensor assembly positioned within the medical sheath assembly. The medical sheath assembly includes a sheath-support component. Preferably, the sheath-support component does not interfere (in a significant way) with the transmission (conveyance) of electromagnetic energy traveling through the medical sheath assembly. Preferably, the sheath-support component is sympathetic (compatible), at least in part, for transmission (conveyance) of electromagnetic energy traveling (moving) through the sheath-support component, dispatched toward, or dispatched away from, the medical sheath assembly.

To mitigate, at least in part, at least one problem associated with the existing technology, there is provided (in accordance with a major aspect) an apparatus. The apparatus is for use with an electro-anatomical mapping system configured to transmit an electromagnetic-transmission signal to a sensor assembly. The sensor assembly is configured to transmit a detected signal back to the electro-anatomical mapping system, in response to the sensor assembly, in use, receiving, and interacting with, electromagnetic-transmission signal emanating from the electro-anatomical mapping system. The apparatus includes and is not limited to (comprises) a medical sheath assembly configured to be positioned in a patient. The medical sheath assembly defines a lumen extending, at least in part, therealong. The lumen is configured to receive the sensor assembly therein. The lumen is also configured to permit movement of the sensor assembly along, at least in part, a length of the lumen. The medical sheath assembly is configured to convey, at least in part, the electromagnetic-transmission signal emanating from the electro-anatomical mapping system through, at least in part, the medical sheath assembly, and toward the sensor assembly positioned within the lumen. This is done in such a way that the medical sheath assembly permits the sensor assembly to transmit the detected signal back to the electro-anatomical mapping system. The medical sheath assembly includes a sheath-support component. Preferably, the sheath-support component does not interfere (in a significant way) with the transmission (conveyance) of electromagnetic energy traveling through the medical sheath assembly. Preferably, the sheath-support component is sympathetic (compatible), at least in part, for transmission (conveyance) of electromagnetic energy traveling (moving) through the sheath-support component, dispatched toward, or dispatched away from, the medical sheath assembly.

To mitigate, at least in part, at least one problem associated with the existing technology, there is provided (in accordance with a major aspect) an apparatus. The apparatus includes and is not limited to (comprises) an electro-anatomical mapping system configured to transmit an electromagnetic-transmission signal. A sensor assembly is configured to receive the electromagnetic-transmission signal emanating from the electro-anatomical mapping system. The sensor assembly is also configured to transmit a detected signal back to the electro-anatomical mapping system, in response to the sensor assembly, in use, receiving, and interacting with, electromagnetic-transmission signal provided by the electro-anatomical mapping system. The apparatus includes and is not limited to (comprises) a medical sheath assembly configured to be positioned in a patient. The medical sheath assembly defines a lumen extending, at least in part, therealong. The lumen is configured to receive the sensor assembly therein. The lumen is also configured to permit movement of the sensor assembly along, at least in part, a length of the lumen. The medical sheath assembly is configured to convey, at least in part, the electromagnetic-transmission signal emanating from the electro-anatomical mapping system through, at least in part, the medical sheath assembly, and toward the sensor assembly positioned within the lumen. This is done in such a way that the medical sheath assembly permits the sensor assembly to transmit the detected signal back to the electro-anatomical mapping system. The medical sheath assembly includes a sheath-support component.

Preferably, the sheath-support component does not interfere (in a significant way) with the transmission (conveyance) of electromagnetic energy traveling through the medical sheath assembly. Preferably, the sheath-support component is sympathetic (compatible), at least in part, for transmission (conveyance) of electromagnetic energy traveling (moving) through the sheath-support component, dispatched toward, or dispatched away from, the medical sheath assembly.

To mitigate, at least in part, at least one problem associated with the existing technology, there is provided (in accordance with a major aspect) a method. The method is for utilizing an electro-anatomical mapping system. The method includes and is not limited to (comprises) operation (A) including utilizing the electro-anatomical mapping system to transmit an electromagnetic-transmission signal. The method also includes operation (B) including utilizing a sensor assembly to receive the electromagnetic-transmission signal emanating from the electro-anatomical mapping system. The method also includes operation (C) including utilizing the sensor assembly to transmit a detected signal back to the electro-anatomical mapping system, in response to the sensor assembly, in use, receiving, and interacting with, electromagnetic-transmission signal provided by the electro-anatomical mapping system. The method also includes operation (D) including positioning a medical sheath assembly in a patient, in which the medical sheath assembly defines a lumen extending, at least in part, therealong, and in which the lumen is configured to receive the sensor assembly therein, and in which the lumen is also configured to permit movement of the sensor assembly along, at least in part, a length of the lumen. The method also includes operation (E) including utilizing the medical sheath assembly to convey, at least in part, the electromagnetic-transmission signal emanating from the electro-anatomical mapping system through, at least in part, the medical sheath assembly, and toward the sensor assembly positioned within the lumen (this is done in such a way that the medical sheath assembly permits the sensor assembly to transmit the detected signal back to the electro-anatomical mapping system, in which the medical sheath assembly includes a sheath-support component positioned to the medical sheath assembly, and in which the sheath-support component is configured to support, at least in part, an attribute of the medical sheath assembly). The method also includes operation (F) including utilizing the sheath-support component to convey, at least in part, the electromagnetic-transmission signal emanating from the electro-anatomical mapping system through, at least in part, the sheath-support component, and toward the sensor assembly positioned within the lumen in such a way that the sheath-support component permits the sensor assembly to transmit the detected signal back to the electro-anatomical mapping system. Preferably, the sheath-support component does not interfere (in a significant way) with the transmission (conveyance) of electromagnetic energy traveling through the medical sheath assembly. Preferably, the sheath-support component is sympathetic (compatible), at least in part, for transmission (conveyance) of electromagnetic energy traveling (moving) through the sheath-support component, dispatched toward, or dispatched away from, the medical sheath assembly.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a schematic view of a medical sheath assembly; and

FIG. 2 depicts a close-up side view of the medical sheath assembly of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

medical sheath assembly 102
lumen 104
sheath-support component 106
distal tip portion 108
patient 800
sensor assembly 900
electro-anatomical mapping system 902
sensor-interface system 904
biological wall 906
medical device 908

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

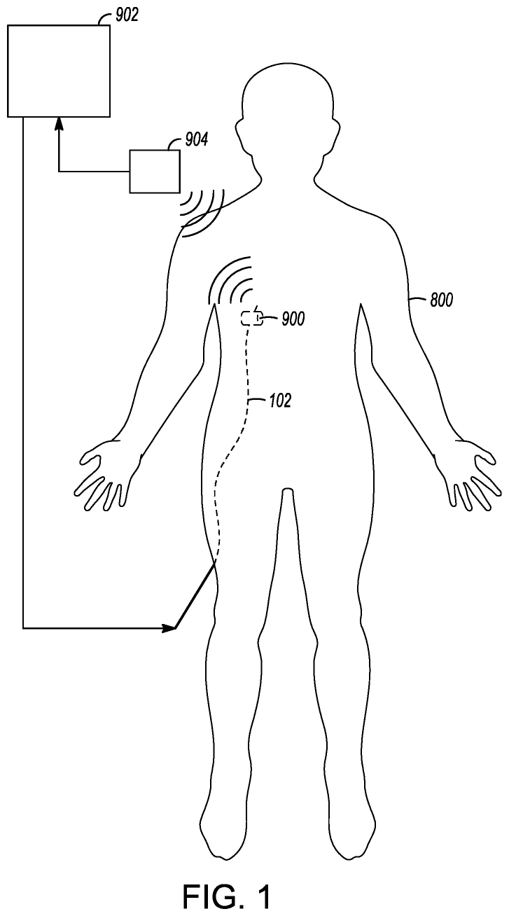

FIG. 1 depicts a schematic view of a medical sheath assembly 102.

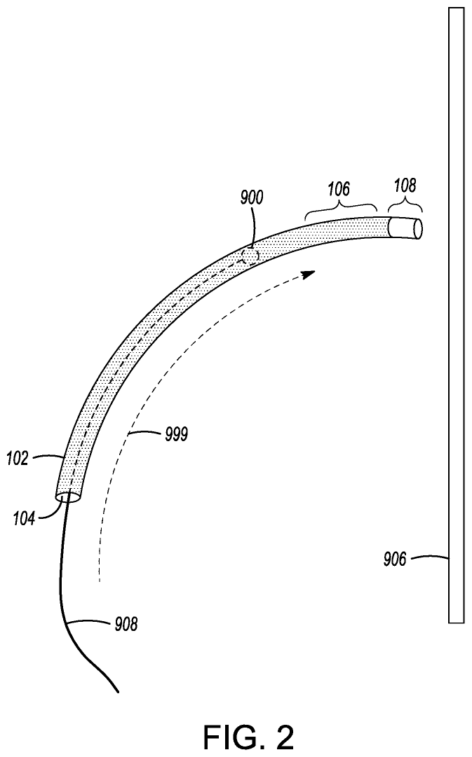

FIG. 2 depicts a close-up side view of the medical sheath assembly 102 of FIG. 1.

Referring to the embodiment as depicted in FIG. 1, an electro-anatomical mapping system 902 is depicted. The electro-anatomical mapping system 902 may include any type of medical signal-measurement system configured to detect (view) any type of medical procedure, medical signal-analysis system, an electromagnetic medical system, an electro-anatomic nonfluoroscopic mapping system, a dielectric imaging system, and/or any type of medical imaging-system (that is, any type of medical imaging-system configured to exchange electromagnetic-transmission signals as an arrangement for interacting with a medical device, such as a sheath assembly), etc., and any equivalent thereof (whether implemented for three-dimensional (3D) viewing and/or two-dimensional (2D) viewing). The electro-anatomical mapping system 902 is configured to be electrically connectable (selectively electrically connectable, coupled) to a sensor-interface system 904 configured to transmit a signal (also called an electromagnetic-transmission signal) to a sensor assembly 900 positioned in a patient 800. The definition of “electrically connected” includes electromagnetically connected, magnetically connected, acoustically connected, photonically connected, etc. The sensor-interface system 904 is configured to electromagnetically communicate (interact) with the sensor assembly 900 (also called a receive sensor, etc.). For instance, the sensor-interface system 904 is configured to exchange (receive and/or transmit) signals (information) with the sensor assembly 900. The exchange of signals may include having the sensor-interface system 904 transmit a signal to the sensor assembly 900 and/or receive a signal from the sensor assembly 900.

Referring to the embodiments as depicted in FIG. 1 and FIG. 2, a medical sheath assembly 102 is configured to be positioned in a patient 800. The medical sheath assembly 102 is also configured to convey (permit the passage of), at least in part, an electromagnetic-transmission signal through the medical sheath assembly 102. The medical sheath assembly 102 includes a sheath-support component 106 (which is depicted in the embodiment of FIG. 2). The sheath-support component 106 is (preferably) configured to convey (permit the passage of), at least in part, the electromagnetic-transmission signal through the sheath-support component 106. Preferably, the sheath-support component 106 does not interfere (in a significant way) with the transmission (conveyance) of electromagnetic energy traveling through the medical sheath assembly 102. Preferably, the sheath-support component 106 is sympathetic (compatible), at least in part, for transmission (conveyance) of electromagnetic energy traveling (moving) through the sheath-support component 106, dispatched toward, or dispatched away from, the medical sheath assembly 102. A technical result of this arrangement is that the electromagnetic energy (which travels (moves) through the sheath-support component 106) may be transmitted from, and/or received by, the electro-anatomical mapping system 902 (as depicted in FIG. 1).

Referring to the embodiments as depicted in FIG. 1 and FIG. 2, the medical sheath assembly 102 includes (preferably) biocompatible material properties suitable for sufficient performance properties (dielectric strength, thermal performance, insulation and corrosion, water and heat resistance) for safe performance to comply with industrial and regulatory safety standards (or compatible for medical usage). Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiments as depicted in FIG. 1 and FIG. 2, a sensor assembly 900 is configured to receive an electromagnetic-transmission signal emanating from an electro-anatomical mapping system 902. The sensor assembly 900 is also configured to transmit a detected signal back to the electro-anatomical mapping system 902. A medical sheath assembly 102 is configured to be positioned in a patient 800. The medical sheath assembly 102 is also configured to receive the sensor assembly 900 therein. The medical sheath assembly 102 is also configured to convey (permit the passage of), at least in part, the electromagnetic-transmission signal emanating from the electro-anatomical mapping system 902 toward the sensor assembly 900 positioned within the medical sheath assembly 102. The medical sheath assembly 102 includes a sheath-support component 106. The sheath-support component 106 is (preferably configured to support (provide improvement for structural strength, stiffness), at least in part, an attribute (structural strength, stiffness) of the medical sheath assembly 102. For instance, the sheath-support component 106 is configured to provide improvement for structural strength of the medical sheath assembly 102. The sheath-support component 106 is (preferably) configured to convey (permit the passage of), at least in part, the electromagnetic-transmission signal emanating from the electro-anatomical mapping system 902 through the sheath-support component 106 and toward the sensor assembly 900 positioned within the medical sheath assembly 102. Preferably, the sheath-support component 106 does not interfere (in a significant way) with the transmission (conveyance) of electromagnetic energy traveling through the medical sheath assembly 102. Preferably, the sheath-support component 106 is sympathetic (compatible), at least in part, for transmission (conveyance) of electromagnetic energy traveling (moving) through the sheath-support component 106, dispatched toward, or dispatched away from, the medical sheath assembly 102. A technical result of this arrangement is that the electromagnetic energy (which travels (moves) through the sheath-support component 106) may be transmitted from, and/or received by, the electro-anatomical mapping system 902 (as depicted in FIG. 1).

Referring to the embodiments as depicted in FIG. 1 and FIG. 2, an electro-anatomical mapping system 902 is configured to transmit an electromagnetic-transmission signal. A sensor assembly 900 is configured to receive the electromagnetic-transmission signal emanating from the electro-anatomical mapping system 902. The sensor assembly 900 is also configured to transmit a detected signal back to the electro-anatomical mapping system 902. A medical sheath assembly 102 is configured to be positioned in a patient 800 (as depicted in FIG. 1). The medical sheath assembly 102 is also configured to receive the sensor assembly 900 therein. The medical sheath assembly 102 is also configured to convey (permit the passage of), at least in part, the electromagnetic-transmission signal emanating from the electro-anatomical mapping system 902 toward the sensor assembly 900 positioned within the medical sheath assembly 102. A medical sheath assembly 102 includes a sheath-support component 106. The sheath-support component 106 is (preferably) configured to support (provide improvement for structural strength, stiffness), at least in part, an attribute (structural strength, stiffness) of the medical sheath assembly 102. The sheath-support component 106 is configured to convey (permit the passage of), at least in part, the electromagnetic-transmission signal emanating from the electro-anatomical mapping system 902. The sheath-support component 106 is (preferably) configured to convey (permit the passage of), at least in part, the electromagnetic-transmission signal emanating from the electro-anatomical mapping system 902 through the sheath-support component 106 toward the sensor assembly 900 positioned within the medical sheath assembly 102. Preferably, the sheath-support component 106 does not interfere (in a significant way) with the transmission (conveyance) of electromagnetic energy traveling through the medical sheath assembly 102. Preferably, the sheath-support component 106 is sympathetic (compatible), at least in part, for transmission (conveyance) of electromagnetic energy traveling (moving) through the sheath-support component 106, dispatched toward, or dispatched away from, the medical sheath assembly 102. A technical result of this arrangement is that the electromagnetic energy (which travels (moves) through the sheath-support component 106) may be transmitted from, and/or received by, the electro-anatomical mapping system 902 (as depicted in FIG. 1).

Referring to the embodiments as depicted in FIG. 1 and FIG. 2, an electro-anatomical mapping system 902 is configured to transmit an electromagnetic-transmission signal to a sensor assembly 900. The sensor assembly 900 is configured to transmit a detected signal back to the electro-anatomical mapping system 902, in response to the sensor assembly 900, in use, receiving, and interacting with, electromagnetic-transmission signal emanating from the electro-anatomical mapping system 902. A medical sheath assembly 102 is configured to be positioned in a patient 800. The medical sheath assembly 102 is (preferably) non-metallic, relatively flexible and elongated. The medical sheath assembly 102 defines a lumen 104 extending, at least in part, therealong (along an elongated length of the medical sheath assembly 102). The lumen 104 is configured to receive the sensor assembly 900 therein. The lumen 104 is also configured to permit movement of the sensor assembly 900 along, at least in part, a length of the lumen 104. The medical sheath assembly 102 is configured to convey (permit the passage of), at least in part, the electromagnetic-transmission signal emanating from the electro-anatomical mapping system 902 through, at least in part, the medical sheath assembly 102, and toward the sensor assembly 900 positioned within the lumen 104. This is done in such a way that the medical sheath assembly 102 permits the sensor assembly 900 to transmit the detected signal back to the electro-anatomical mapping system 902. The medical sheath assembly 102 includes a sheath-support component 106. The sheath-support component 106 includes a braid assembly positioned to the medical sheath assembly 102. The sheath-support component 106 is configured to support (provide improvement for structural strength, stiffness), at least in part, an attribute (structural strength, stiffness) of the medical sheath assembly 102. The sheath-support component 106 is configured to convey (permit the passage of), at least in part, the electromagnetic-transmission signal emanating from the electro-anatomical mapping system 902 through, at least in part, the sheath-support component 106. The sheath-support component 106 is configured to convey (permit the passage of), at least in part, the electromagnetic-transmission signal emanating from the electro-anatomical mapping system 902 through, at least in part, the sheath-support component 106, and toward the sensor assembly 900 positioned within the lumen 104; this is done in such a way that the sheath-support component 106 permits the sensor assembly 900 to transmit the detected signal back to the electro-anatomical mapping system 902. Preferably, the sheath-support component 106 does not interfere (in a significant way) with the transmission (conveyance) of electromagnetic energy traveling through the medical sheath assembly 102. Preferably, the sheath-support component 106 is sympathetic (compatible), at least in part, for transmission (conveyance) of electromagnetic energy traveling (moving) through the sheath-support component 106, dispatched toward, or dispatched away from, the medical sheath assembly 102. A technical result of this arrangement is that the electromagnetic energy (which travels (moves) through the sheath-support component 106) may be transmitted from, and/or received by, the electro-anatomical mapping system 902 (as depicted in FIG. 1).

Referring to the embodiments as depicted in FIG. 1 and FIG. 2, an electro-anatomical mapping system 902 is configured to transmit an electromagnetic-transmission signal. A sensor assembly 900 is configured to receive the electromagnetic-transmission signal emanating from the electro-anatomical mapping system 902. The sensor assembly 900 is also configured to transmit a detected signal back to the electro-anatomical mapping system 902, in response to the sensor assembly 900, in use, receiving, and interacting with, electromagnetic-transmission signal provided by the electro-anatomical mapping system 902. A medical sheath assembly 102 is configured to be positioned in a patient 800. The medical sheath assembly 102 defines a lumen 104 extending, at least in part, therealong. The lumen 104 is configured to receive the sensor assembly 900 therein. The lumen 104 is also configured to permit movement of the sensor assembly 900 along, at least in part, a length of the lumen 104. The medical sheath assembly 102 is configured to convey (permit the passage of), at least in part, the electromagnetic-transmission signal emanating from the electro-anatomical mapping system 902 through, at least in part, the medical sheath assembly 102, and toward the sensor assembly 900 positioned within the lumen 104 in such a way that the medical sheath assembly 102 permits the sensor assembly 900 to transmit the detected signal back to the electro-anatomical mapping system 902. The medical sheath assembly 102 includes a sheath-support component 106. The sheath-support component 106 is (preferably) positioned to (in or on) the medical sheath assembly 102. The sheath-support component 106 is (preferably) configured to support (provide improvement for structural strength, stiffness), at least in part, an attribute (structural strength, stiffness) of the medical sheath assembly 102. The sheath-support component 106 is (preferably) configured to convey (permit the passage of), at least in part, the electromagnetic-transmission signal. The sheath-support component 106 is (preferably) configured to convey (permit the passage of), at least in part, the electromagnetic-transmission signal emanating from the electro-anatomical mapping system 902 through, at least in part, the sheath-support component 106, and toward the sensor assembly 900 positioned within the lumen 104 (this is done in such a way that the sheath-support component 106 permits the sensor assembly 900 to transmit the detected signal back to the electro-anatomical mapping system 902). Preferably, the sheath-support component 106 does not interfere (in a significant way) with the transmission (conveyance) of electromagnetic energy traveling through the medical sheath assembly 102. Preferably, the sheath-support component 106 is sympathetic (compatible), at least in part, for transmission (conveyance) of electromagnetic energy traveling (moving) through the sheath-support component 106, dispatched toward, or dispatched away from, the medical sheath assembly 102. A technical result of this arrangement is that the electromagnetic energy (which travels (moves) through the sheath-support component 106) may be transmitted from, and/or received by, the electro-anatomical mapping system 902 (as depicted in FIG. 1).

Referring to the embodiments as depicted in FIG. 1 and FIG. 2, there is provided a method of utilizing an electro-anatomical mapping system 902. The method includes utilizing the electro-anatomical mapping system 902 to transmit an electromagnetic-transmission signal. The method also includes utilizing a sensor assembly 900 to receive the electromagnetic-transmission signal emanating from the electro-anatomical mapping system 902. The method also includes utilizing the sensor assembly 900 to transmit a detected signal back to the electro-anatomical mapping system 902, in response to the sensor assembly 900, in use, receiving, and interacting with, electromagnetic-transmission signal provided by the electro-anatomical mapping system 902. The method also includes positioning a medical sheath assembly 102 in a patient 800, in which the medical sheath assembly 102 defines a lumen 104 extending, at least in part, therealong, and in which the lumen 104 is configured to receive the sensor assembly 900 therein, and in which the lumen 104 is also configured to permit movement of the sensor assembly 900 along, at least in part, a length of the lumen 104. The method also includes utilizing the medical sheath assembly 102 to convey (permit the passage of), at least in part, the electromagnetic-transmission signal emanating from the electro-anatomical mapping system 902 through, at least in part, the medical sheath assembly 102, and toward the sensor assembly 900 positioned within the lumen 104 in such a way that the medical sheath assembly 102 permits the sensor assembly 900 to transmit the detected signal back to the electro-anatomical mapping system 902, in which the medical sheath assembly 102 includes a sheath-support component 106 positioned to the medical sheath assembly 102, and in which the sheath-support component 106 is configured to support (such as, provide improvement for structural strength and/or stiffness), at least in part, an attribute (such as structural strength and/or stiffness) of the medical sheath assembly 102. The method also includes utilizing the sheath-support component 106 to convey (permit the passage of), at least in part, the electromagnetic-transmission signal emanating from the electro-anatomical mapping system 902 through, at least in part, the sheath-support component 106, and toward the sensor assembly 900 positioned within the lumen 104 (this is one in such a way that the sheath-support component 106 permits the sensor assembly 900 to transmit the detected signal back to the electro-anatomical mapping system 902). Preferably, the sheath-support component 106 does not interfere (in a significant way) with the transmission (conveyance) of electromagnetic energy traveling through the medical sheath assembly 102. Preferably, the sheath-support component 106 is sympathetic (compatible), at least in part, for transmission (conveyance) of electromagnetic energy traveling (moving) through the sheath-support component 106, dispatched toward, or dispatched away from, the medical sheath assembly 102. A technical result of this arrangement is that the electromagnetic energy (which travels (moves) through the sheath-support component 106) may be transmitted from, and/or received by, the electro-anatomical mapping system 902 (as depicted in FIG. 1).

Referring to the embodiments as depicted in FIG. 1 and FIG. 2, the medical sheath assembly 102 may include any type of medical device, such as an intravascular device, etc., and any equivalent thereof. The medical sheath assembly 102 may include any type of intravascular device defining (having) a lumen, etc., and any equivalent thereof. The medical sheath assembly 102 may be configured to be inserted (at least in part) intravascularly to guide a medical device to a target location (such as a target location positioned in the heart of the patient, etc.), and to protect a biological wall 906 from contact with the medical device 908 (the medical device 908 is moveable along the interior of the medical sheath assembly 102 toward the biological wall 906). The biological wall 906 may include, for instance, the endothelium, which is the tissue that forms a single layer of cells lining various organs and cavities of the body, especially the blood vessels, heart, and lymphatic vessels (the endothelium is formed from the embryonic mesoderm).

Referring to the embodiments as depicted in FIG. 1 and FIG. 2, the sheath-support component 106 may include (preferably) a non-metal braid material configured to allow, at least in part, the movement of electromagnetic fields to pass (to bi-directionally pass) through the sheath-support component 106. For instance, the non-metal braid may include (preferably) a PolyEtherEther-Ketone polymer (PEEK). The sheath-support component 106 is configured to provide improved (additional) structural support for the medical sheath assembly 102. The non-metal braid material may include any type of interlaced structure. The sheath-support component 106 is configured to improve (at least in part) a structural aspect or attribute of the medical sheath assembly 102 (such as, the burst pressure resistance, column strength, and/or torque transmission, etc., and any equivalent thereof). The sheath-support component 106 may have a braid pattern that may vary.

Referring to the embodiments as depicted in FIG. 1 and FIG. 2, the sheath-support component 106 is configured to permit spatial tracking of the sensor assembly 900 by the electro-anatomical mapping system 902. The sensor assembly 900 is mounted to, for instance, the medical device 908. The medical device 908 may include a catheter device, etc., and any equivalent thereof. The sensor assembly 900 may include any type of sensor, such as an electrode (any electrode configured to exchange electromagnetic-transmission signals as an arrangement for interaction), a coil, an antenna, etc., and any equivalent thereof. The sensor assembly 900 may be detected by the electro-anatomical mapping system 902 while the sensor assembly 900 is positioned in the lumen 104 of the medical sheath assembly 102. The sensor assembly 900 is configured to exchange electromagnetic-transmission signals as an arrangement for interaction.

Referring to the embodiments as depicted in FIG. 1 and FIG. 2, the medical sheath assembly 102 includes an exposed distal tip portion 108 that is devoid of the sheath-support component 106. The exposed distal tip portion 108 is relatively softer than section of the medical sheath assembly 102 having the sheath-support component 106. The exposed distal tip portion 108 is configured to avoid harming the biological wall 906 (for the case where the exposed distal tip portion 108 is moved to contact the biological wall 906).

Referring to the embodiments as depicted in FIG. 1 and FIG. 2, the sheath-support component 106 (preferably) may include a non-metallic material. The sheath-support component 106 may include a metallic material. The sheath-support component 106 may be segmented into metallic sections and non-metallic sections (if desired). It will be appreciated that there may be types of electromagnetic waves that may penetrate a metallic material. Relatively lower frequencies may penetrate a metallic material. Electromagnetic waves (energy) contain oscillating electrical fields and these fields may disturb the electrons in conductors and produce a penetration of an electric field into the conductor. This is called the skin effect. The longer the wavelength of the incident electromagnetic wave, the deeper the penetration in a metallic material. Even at low frequencies, there is a finite depth of penetration of electromagnetic waves in metals because they are not perfect conductors with infinite conductivity.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for use with a sensor assembly configured to receive an electromagnetic-transmission signal being emanated from an electro-anatomical mapping system, and the sensor assembly also configured to transmit a detected signal back to the electro-anatomical mapping system, the apparatus comprising:
a medical sheath assembly configured to be positioned in a patient, and also configured to receive the sensor assembly therein, and also configured to convey, at least in part, the electromagnetic-transmission signal being emanated from the electro-anatomical mapping system toward the sensor assembly positioned within the medical sheath assembly; and
the medical sheath assembly including a sheath-support component being sympathetic, at least in part, for transmission of electromagnetic energy traveling through the sheath-support component, dispatched toward, or dispatched away from, the medical sheath assembly,
wherein:
the sheath-support component is configured to support, at least in part, an attribute of the medical sheath assembly and
to provide improvement for structural strength of the medical sheath assembly.

2. The apparatus of claim 1, wherein:
the sheath-support component configured to convey, at least in part, the electromagnetic-transmission signal being emanated from the electro-anatomical mapping system through the sheath-support component and toward the sensor assembly positioned within the medical sheath assembly.

3. An apparatus, comprising:
an electro-anatomical mapping system configured to transmit an electromagnetic-transmission signal; and
a sensor assembly configured to receive the electromagnetic-transmission signal being emanating from the electro-anatomical mapping system; and
the sensor assembly also configured to transmit a detected signal back to the electro-anatomical mapping system, in response to the sensor assembly, in use, receiving, and interacting with, electromagnetic-transmission signal provided by the electro-anatomical mapping system, the apparatus comprising:
a medical sheath assembly configured to be positioned in a patient; and
the medical sheath assembly defining a lumen extending, at least in part, therealong; and
the lumen configured to receive the sensor assembly therein, and the lumen also configured to permit movement of the sensor assembly along, at least in part, a length of the lumen; and
the medical sheath assembly configured to convey, at least in part, the electromagnetic-transmission signal being emanated from the electro-anatomical mapping system through, at least in part, the medical sheath assembly, and toward the sensor assembly positioned within the lumen in such a way that the medical sheath assembly permits the sensor assembly to transmit the detected signal back to the electro-anatomical mapping system; and
the medical sheath assembly including a sheath-support component being sympathetic, at least in part, for transmission of electromagnetic energy traveling through the sheath-support component, dispatched toward, or dispatched away from, the medical sheath assembly.

4. The apparatus of claim 3, wherein:
the sheath-support component is positioned to the medical sheath assembly.

5. The apparatus of claim 4, wherein:
the sheath-support component is configured to support, at least in part, an attribute of the medical sheath assembly.

6. The apparatus of claim 5, wherein:
the sheath-support component is configured to convey, at least in part, the electromagnetic-transmission signal.

7. The apparatus of claim 5, wherein:
the sheath-support component is configured to convey, at least in part, the electromagnetic-transmission signal being emanated from the electro-anatomical mapping system through, at least in part, the sheath-support component, and toward the sensor assembly positioned within the lumen in such a way that the sheath-support component permits the sensor assembly to transmit the detected signal back to the electro-anatomical mapping system.

8. The apparatus of claim 3, wherein:
the medical sheath assembly includes an intravascular device.

9. The apparatus of claim 3, wherein:
the sheath-support component includes a non-metallic material.

10. The apparatus of claim 3, wherein:
the sheath-support component is configured to permit spatial tracking of the sensor assembly by the electro-anatomical mapping system.

11. The apparatus of claim 3, wherein:
the sheath-support component includes a metallic material.

12. The apparatus of claim 3, wherein:
the medical sheath assembly includes an exposed distal tip portion that is devoid of the sheath-support component; and
the exposed distal tip portion is relatively softer than section of the medical sheath assembly having the sheath-support component.

13. A method of utilizing an electro-anatomical mapping system, the method comprising:
utilizing the electro-anatomical mapping system to transmit an electromagnetic-transmission signal;
utilizing a sensor assembly to receive the electromagnetic-transmission signal being emanated from the electro-anatomical mapping system;
utilizing the sensor assembly to transmit a detected signal back to the electro-anatomical mapping system, in response to the sensor assembly, in use, receiving, and interacting with, electromagnetic-transmission signal provided by the electro-anatomical mapping system;
positioning a medical sheath assembly in a patient, in which the medical sheath assembly defines a lumen extending, at least in part, therealong, and in which the lumen is configured to receive the sensor assembly therein, and in which the lumen is also configured to permit movement of the sensor assembly along, at least in part, a length of the lumen;
utilizing the medical sheath assembly to convey, at least in part, the electromagnetic-transmission signal being emanated from the electro-anatomical mapping system through, at least in part, the medical sheath assembly, and toward the sensor assembly positioned within the lumen in such a way that the medical sheath assembly permits the sensor assembly to transmit the detected signal back to the electro-anatomical mapping system, in which the medical sheath assembly includes a sheath-support component positioned to the medical sheath assembly, and in which the sheath-support component is configured to support, at least in part, an attribute of the medical sheath assembly, and in which the sheath-support component is sympathetic, at least in part, for transmission of electromagnetic energy traveling through the sheath-support component, dispatched toward, or dispatched away from, the medical sheath assembly; and
utilizing the sheath-support component to convey, at least in part, the electromagnetic-transmission signal being emanated from the electro-anatomical mapping system through, at least in part, the sheath-support component, and toward the sensor assembly positioned within the lumen in such a way that the sheath-support component permits the sensor assembly to transmit the detected signal back to the electro-anatomical mapping system.

14. The method of claim 13, wherein:
the medical sheath assembly includes an exposed distal tip portion that is devoid of the sheath-support component; and
the exposed distal tip portion is relatively softer than section of the medical sheath assembly having the sheath-support component.

15. The method of claim 13, wherein:
the medical sheath assembly includes an intravascular device.

16. The method of claim 13, wherein:
the sheath-support component includes a non-metallic material.

17. The method of claim 13, wherein:
the sheath-support component is configured to permit spatial tracking of the sensor assembly by the electro-anatomical mapping system.

18. The method of claim 13, wherein:
the sheath-support component includes a metallic material.
